(19) United States
(12) Patent Application Publication
(10) Pub. No.: US 2024/0161902 A1
(43) Pub. Date: May 16, 2024

(54) MULTI-CAMERA MACHINE LEARNING VIEW TRACKING

(71) Applicant: NEC Laboratories America, Inc., Princeton, NJ (US)

(72) Inventors: Deep Patel, Franklin Park, NJ (US); Alexandru Niculescu-Mizil, Plainsboro, NJ (US); Iain Melvin, Princeton, NJ (US); Seonghyeon Moon, Piscataway, NJ (US)

(21) Appl. No.: 18/505,761

(22) Filed: Nov. 9, 2023

Related U.S. Application Data
(60) Provisional application No. 63/424,517, filed on Nov. 11, 2022.
(60) Provisional application No. 63/464,247, filed on May 5, 2023.

(51) Int. Cl. G16H 20/30; G06T 7/292; G16H 50/20

(52) U.S. Cl. CPC G16H 20/30; G06T 7/292; G16H 50/20; G06T 2207/10016; G06T 2207/30196

(57) ABSTRACT

Methods and systems for tracking movement include performing person detection in frames from multiple video streams to identify detection images. Visual and location information from the detection images are combined to generate scores for pairs of detection images across the multiple video streams and across frames of respective video streams. A pairwise detection graph is generated using the detection images as nodes and the scores as weighted edges. A current view of the multiple video streams is changed to a next view of the multiple video streams, responsive to a determination that a score between consecutive frames of the view is below a threshold value and that a score between coincident frames of the current view and the next view is above the threshold value.

### RELATED APPLICATION INFORMATION

This application claims priority to U.S. Patent Application No. 63/424,517, filed on Nov. 11, 2022, and to U.S. Patent Application No. 63/464,247, filed May 5, 2023, each incorporated herein by reference in its entirety. This application is related to an application entitled “MACHINE LEARNING MODEL FOR MULTI-CAMERA MULTI-PERSON TRACKING”, having attorney docket number 22076, and which is incorporated by reference herein in its entirety.

### BACKGROUND

### Technical Field

The present invention relates to video processing and, more particularly, to tracking individuals across multiple camera.

### Description of the Related Art

Large public spaces may be monitored to provide security, public safety, and healthcare services. Multiple cameras may be installed in a given space to help track individuals as they move through the space. However, there may be some locations within the space without overlapping camera views, which makes it challenging to track a person through those locations, or in situations where occlusions block an image of the person.

### SUMMARY

A method for tracking movement includes performing person detection in frames from multiple video streams to identify detection images. Visual and location information from the detection images are combined to generate scores for pairs of detection images across the multiple video streams and across frames of respective video streams. A pairwise detection graph is generated using the detection images as nodes and the scores as weighted edges. A current view of the multiple video streams is changed to a next view of the multiple video streams, responsive to a determination that a score between consecutive frames of the view is below a threshold value and that a score between coincident frames of the current view and the next view is above the threshold value.

A system for tracking movement includes a hardware processor and a memory that stores a computer program. When executed by the hardware processor, the computer program causes the hardware processor to perform person detection in frames from multiple video streams to identify detection images, to combine visual and location information from the detection images to generate scores for pairs of detection images across the multiple video streams and across frames of respective video streams, to generate a pairwise detection graph using the detection images as nodes and the scores as weighted edges, and to change a current view of the multiple video streams to a next view of the multiple video streams, responsive to a determination that a score between consecutive frames of the view is below a threshold value and that a score between coincident frames of the current view and the next view is above the threshold value.

A method for tracking movement in a healthcare facility includes performing person detection in frames from multiple video streams in a healthcare facility to identify detection images. Visual and location information from the detection images are combined to generate scores for pairs of detection images across the multiple video streams and across frames of respective video streams. A pairwise detection graph is generated using the detection images as nodes and the scores as weighted edges. Movement of a patient is tracked based on a selected detection image and a comparison of the scores to a threshold value. A current view of the multiple video streams is changed to a next view of the multiple video streams, responsive to a determination that a score between consecutive frames of the view is below a threshold value and that a score between coincident frames of the current view and the next view is above the threshold value. A report is generated for a healthcare professional for decision-making related to a patient's treatment, based on the tracked movement.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram of an environment that is monitored by multiple video cameras, in accordance with an embodiment of the present invention;

FIG. 2 is a block/flow diagram of a method for tracking movement of an individual using multiple video cameras, in accordance with an embodiment of the present invention;

FIG. 3 is a diagram illustrating relationships between detected person images between frames of a given video stream and between different video streams, in accordance with an embodiment of the present invention;

FIG. 4 is a block/flow diagram of tracking movement with a preferred camera view, in accordance with an embodiment of the present invention;

FIG. 5 is a block diagram illustrating person tracking within a healthcare facility, in accordance with an embodiment of the present invention;

FIG. 6 is a block/flow diagram of a method for tracking and responding to an individual's movements, in accordance with an embodiment of the present invention;

FIG. 7 is a block diagram of a computing device that can track and respond to an individual's movements, in accordance with an embodiment of the present invention;

FIG. 8 is a diagram illustrating an exemplary neural network architecture that can be used as part of a person detection model, in accordance with an embodiment of the present invention; and

FIG. 9 is a diagram illustrating an exemplary deep neural network architecture that can be used as part of a person detection model, in accordance with an embodiment of the present invention.

### DETAILED DESCRIPTION OF PREFERRED EMBODIMENTS

Visual and positional information may be combined across multiple camera views to aid in tracking individuals through a space. The combined information may be used to obtain a scalar score between pairs of detections across frames and cameras. Using a fully connected graph of all of the detections, with detections being represented as nodes and with the scores being represented as edges, a constraint logic program can be used to find the best associations between each detection.

Visual information and positioning information may be combined from multiple video sources, which avoids visual biases and provides accurate associations, even for occluded individuals. Additionally, in the event that the system makes a mistake in identifying an individual, that mistake can be corrected as further information is collected.

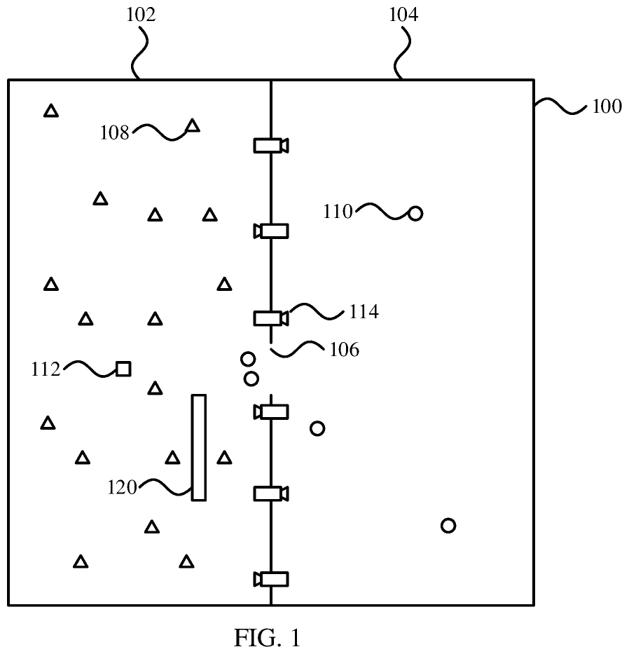

Referring now in detail to the figures in which like numerals represent the same or similar elements and initially to FIG. 1, an environment 100 is shown. For example, one type of environment that is contemplated is a mall or shopping center, which may include a common space 102 and one or more regions 104, such as a store. It should be understood that this example is provided solely for the purpose of illustration, and should not be regarded as limiting.

A boundary is shown between the common space 102 and the region 104. The boundary can be any appropriate physical or virtual boundary. Examples of physical boundaries include walls and rope—anything that establishes a physical barrier to passage from one region to the other. Examples of virtual boundaries include a painted line and a designation within a map of the environment 100. Virtual boundaries do not establish a physical barrier to movement, but can nonetheless be used to identify regions within the environment. For example, a region of interest may be established next to an exhibit or display, and can be used to indicate people's interest in that display. A gate 106 is shown as a passageway through the boundary, where individuals are permitted to pass between the common space 102 and the region 104.

The environment 100 is monitored by a number of video cameras 114. Although this embodiment shows the cameras 114 being positioned at the gate 106, it should be understood that such cameras can be positioned anywhere within the common space 102 and the region 104. The video cameras 114 capture live streaming video of the individuals in the environment. A number of individuals are shown, including untracked individuals 108, shown as triangles, and tracked individuals 110, shown as circles. Also shown is a tracked person of interest 112, shown as a square. In some examples, all of the individuals may be tracked individuals. In some examples, the tracked person of interest 112 may be tracked to provide an interactive experience, with their motion through the environment 100 being used to trigger responses.

In addition to capturing visual information, the cameras 114 may capture other types of data. For example, the cameras 114 may be equipped with infrared sensors that can read the body temperature of an individual. In association with the visual information, this can provide the ability to remotely identify individuals who are sick, and to track their motion through the environment.

The environment 100 may include occlusions 120. For example, fixed occlusions may include walls, staircases, escalators, and other barriers. Movable occlusions may include people, signage, vehicles, and other objects that may make up a dynamic environment. The occlusions 120 may prevent individuals from being visible from certain angles and to certain cameras 114. Thus a person who is visible from one camera may not be visible to another camera, even if their visual ranges otherwise overlap.

Visual and location information may be collected for each tracked person 110 using frames from the video cameras 114. The frames from respective video streams may be synchronized in time, so that different views of the environment 100 may be compared to one another for given points in time.

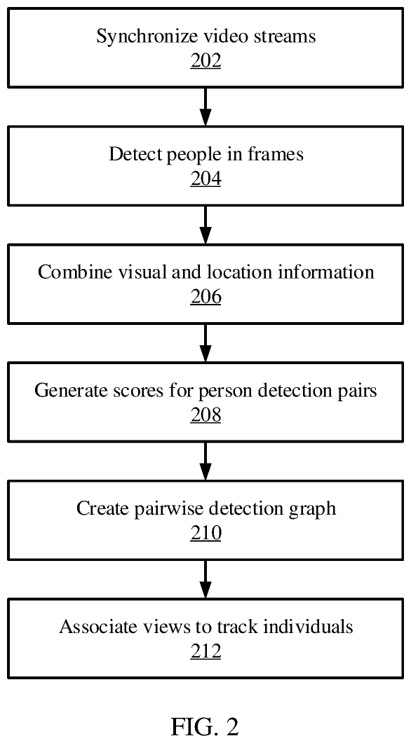

Referring now to FIG. 2, a method for tracking individuals using multiple video streams is shown. Block 202 synchronizes the video streams from multiple cameras 114, so that frames that correspond to one another in time can be identified. This synchronization identifies temporal correspondences between the frames of different video streams. Visual information can be extracted from these frames in block 204 using a person detection model and an image re-identification model. The extracted visual information may include coordinates and a bounding box for each person detected within a given frame. The two-dimensional coordinates of the frame may be projected into a three-dimensional space to determine coordinates for the person within the environment. This projection may be performed using intrinsic and extrinsic parameters of the camera. Thus the frames may be processed along a visual branch and a location branch to generate respective visual and location information.

The visual and location information may be combined in block 206 and a scalar score may be generated for pairs of person images from the frames. For example, two cameras viewing a same scene may each detect three people. In this example, nine scores may be generated to reflect the nine different possible pairings of the person images. Pairs that show the same person may have a higher score than pairs that show different people. These scores may be generated across frames at from different video streams at a given time, and may also be generated across frames from a same video stream at sequential times. Exemplary scoring functions may include cosine similarity and Euclidean distance to identify the distance between visual features and location coordinates, respectively, in the respective image pairs. The distance measures may be summed together to generate the output score.

Block 210 creates a pairwise person detection graph, for example as a fully connected graph that represents person detections as nodes and that represents the scores for a pair of person detection images as a weighted edge between the respective nodes. Block 212 maximizes the overall score of the detection graph while maintaining constraints to match each person node with a unique track. Logical constraints for solving the detection graph may be set for the highest possible score by matching nodes based on their edge scores.

When tracking multiple individuals, constrained answer set programming may be used in block 212. Answer set programming solves the association of persons detected using visual and location scores obtained from block 210. The constraints may include simple logical assumptions, such as a person in the same camera view not having the same track ID as another and minimum matching score thresholds to determine whether two person detections belong to the same track. These constraints may be determined based on the distribution of matching scores and logical assumptions that may be used to find an optimal solution. The optimal solution matches people in different views and frames while satisfying the specified constraints. The output of block 212 may then be the set of tracks that associates all the persons in the detection graph.

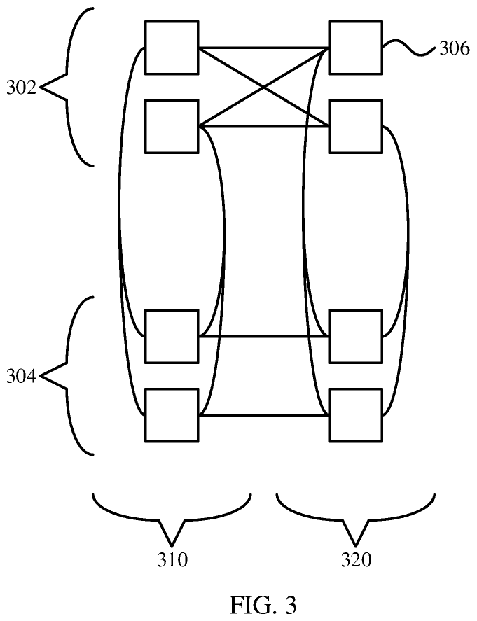

Referring now to FIG. 3, an example of pairwise matching between detected person images is shown. A first video stream 302 and a second video stream 304 are synchronized, so that frames align to particular time stamps. For example, a first time stamp 310 may have a corresponding frame in the first video stream 302 and the second video stream 304, and similarly a second time stamp 320 may have a corresponding frame in each of the video streams.

Person detection is performed on the frames of the video streams. For each frame, detected person images may be extracted, for example with pixels within the bounding box of the detected person. These images may be compared across video streams and across time stamps and a score may be generated for each compared pair. In this manner, associations can be built across multiple views of the environment and across time. If a given person is not visible in a particular video stream, for example because they have moved behind an occlusion or have left the camera's field of view, their presence in other video streams can be used to continue tracking them through the environment.

Thus, for each pair of detection images, a visual comparison and a spatial comparison may be performed. The visual comparison may use a re-identification model to generate visual features from different camera views. The spatial comparison may identify a representative coordinate of each detected image and the distance between these coordinates may be determined. The output of the visual comparison and the spatial comparison may be combined to generate a score between 0 and 1, representing a match between the images of the pair.

The visual comparison may be performed by extracting feature embeddings for the person detection using a re-identification model, such as a residual network (ResNet) or a vision transformer (ViT) network. These models may be pretrained on reidentification datasets to match a person with similar visual features at different viewing angles and in varied lighting conditions. The model may also be trained on a multi-camera tracking dataset. The visual features between a pair of detections can be compared using any appropriate similarity metric, such as cosine similarity or Euclidean distance between feature vectors. The embeddings for the same person may have a smaller distance than embeddings of different people.

For spatial or location comparisons, the position of the person may be projected from the camera views to a global coordinate space. The same person in different camera views will have similar location coordinates in the global space. The camera-to-global projection can be performed using intrinsic and extrinsic parameters of the camera. Spatial comparison can also be learned using a simple transformer model, which can take as input multi-view frames and two-dimensional bounding box coordinates of people in each view. The output of the spatial comparison may include matching scores for each person detection, learned from the projection matrix from the multi-camera tracking datasets.

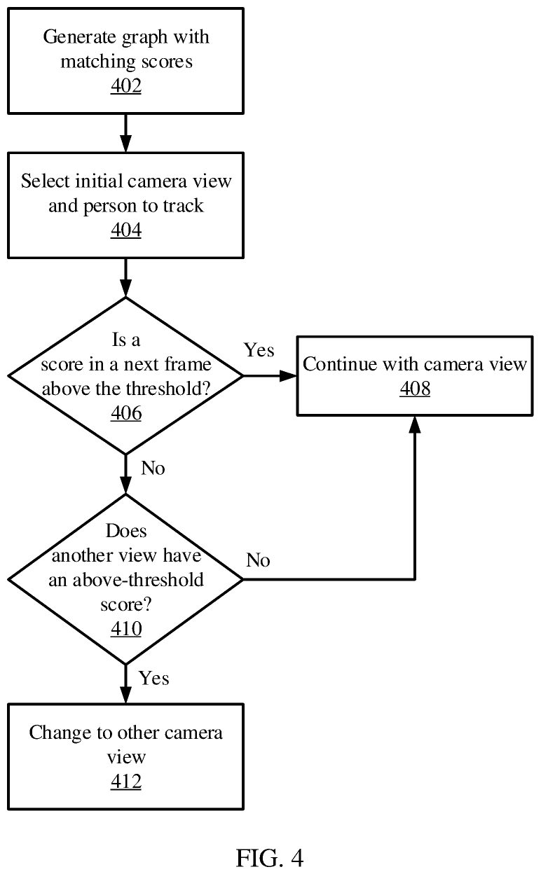

Referring now to FIG. 4, a method for tracking a person across multiple camera views is shown. A person may be tracked through the environment by finding relatively high scores. For example, if a score is higher than a threshold (e.g., greater than 0.9), then an association may be determined between the corresponding pair of images. If there is no high-score pair between subsequent frames of a given video stream, but there is instead a high-score pair between video streams, then a main camera view may be changed. This may reflect a situation where a person has left the view of a camera or has moved behind an occlusion, but remains visible in another video stream.

Thus, block 402 generates a graph with matching scores as described above. The nodes of the graph, representing person detections, may be connected to other person detections in other frames of a given video stream and to other video streams at a given time, with edges being weighted according to their combined visual and location score information.

Block 404 selects an initial camera view and further selects a person detection within the initial camera view to track. Block 406 determines whether a score between the selected person detection and a detection from a subsequent or consecutive frame of the initial camera view is above the threshold. If so, block 408 continues with the current camera view.

If not, block 410 determines whether a score between the selected person detection and a detection from another camera view at the same time is above the threshold, for example looking to coincident frames. If not, block 408 may continue with the current camera view, as there is no matching person image in other video streams. If another view does have an above-threshold score for an image detection, then block 412 changes to the other camera view. In this manner, the selected person may be tracked as they move from one camera view to another and as they pass behind occlusions. The changing of views may be implemented in a user interface that receives multiple video streams and that automatically selects a video stream to display to track a given individual.

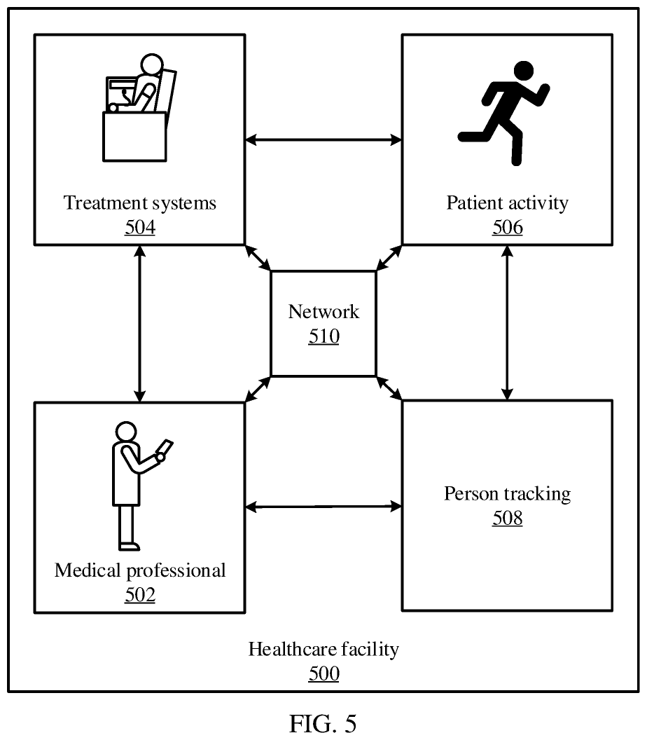

Referring now to FIG. 5, a diagram of action recognition is shown in the context of a healthcare facility 500. As a patient moves through a healthcare facility 500, their activity 506 may be monitored by video cameras installed in the facility 500. Person tracking 508 may be used to monitor patient activity 506, which can help to understand the patient's behavior and healthcare needs. For example, person tracking 508 may provide information on how the patient interacts with treatment systems 504.

The healthcare facility may include one or more medical professionals 502 who provide information relating to the patient's activity and information provided by the treatment systems 504. As person tracking 508 is performed using the patient activity 506, information may be automatically generated regarding the patient's behavior. Behavior that reflects some unmet need or some risk may be included in a report for a medical professional 502 to use in decision making. For example, based on the report, the medical professional 502 may correct the patient's treatment to promote healthy activity.

The different elements of the healthcare facility 500 may communicate with one another via a network 510, for example using any appropriate wired or wireless communications protocol and medium. The medical professional 502 may thereby receive information relating to the patient activity 506 from the person tracking 508.

In other embodiments, tracking may be performed in any heavily occluded or complex environment with multiple cameras, such as museums, shopping malls, and airports. Tracking individuals at heavily crowded events, such as sporting events, can be accomplished using cameras on unmanned aerial vehicles. The tracking can also be used to back-trace the path taken by an individual for offline analytics and other non-real-time investigations.

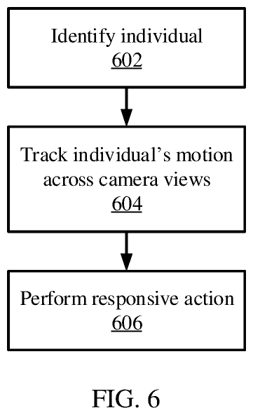

Referring now to FIG. 6, a method of tracking and responding to an individual's movements is shown. Block 602 identifies an individual in a video stream. This individual may be selected in a user interface, and the individual's motion may be tracked across camera views in block 604.

Based on the tracked motion, block 606 performs a responsive action. As described above, the responsive action may include a healthcare-oriented action, such as generating a report to be used in medical decision-making. In some cases, the responsive action may include a security action, such as locking or unlocking a door, permitting or denying access, summoning alerting security personnel. The responsive action may be performed automatically upon detection that the tracked movement has satisfied an appropriate criterion. For example, if the individual is tracked in a place where they are not authorized access, a security action may be automatically triggered. If the individual's movements indicate a negative health event, a healthcare response may be automatically triggered.

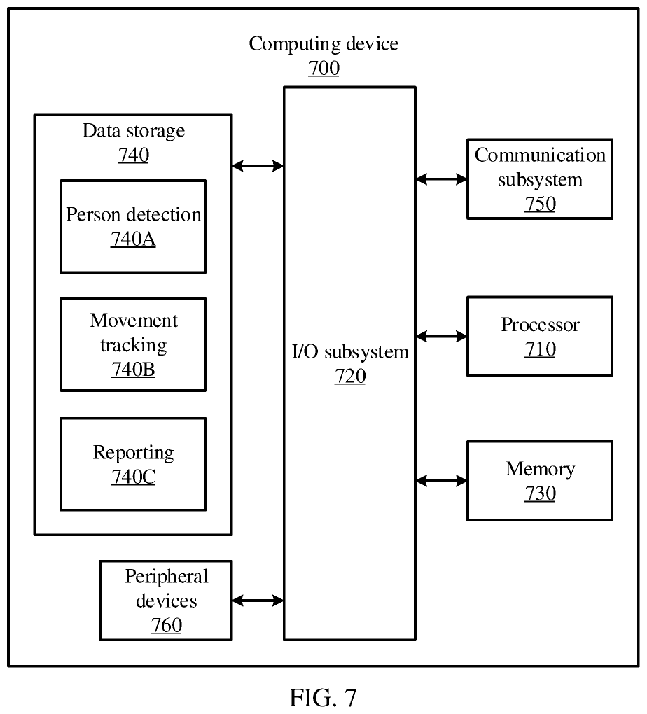

Referring now to FIG. 7, an exemplary computing device 700 is shown, in accordance with an embodiment of the present invention. The computing device 700 is configured to track movement across multiple cameras.

The computing device 700 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 700 may be embodied as one or more compute sleds, memory sleds, or other racks, sleds, computing chassis, or other components of a physically disaggregated computing device.

As shown in FIG. 7, the computing device 700 illustratively includes the processor 710, an input/output subsystem 720, a memory 730, a data storage device 740, and a communication subsystem 750, and/or other components and devices commonly found in a server or similar computing device. The computing device 700 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 730, or portions thereof, may be incorporated in the processor 710 in some embodiments.

The processor 710 may be embodied as any type of processor capable of performing the functions described herein. The processor 710 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 730 may be embodied as any type of volatile or non-volatile memory or data storage capable of performing the functions described herein. In operation, the memory 730 may store various data and software used during operation of the computing device 700, such as operating systems, applications, programs, libraries, and drivers. The memory 730 is communicatively coupled to the processor 710 via the I/O subsystem 720, which may be embodied as circuitry and/or components to facilitate input/output operations with the processor 710, the memory 730, and other components of the computing device 700. For example, the I/O subsystem 720 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 720 may form a portion of a system-on-a-chip (SOC) and be incorporated, along with the processor 710, the memory 730, and other components of the computing device 700, on a single integrated circuit chip.

The data storage device 740 may be embodied as any type of device or devices configured for short-term or long-term storage of data such as, for example, memory devices and circuits, memory cards, hard disk drives, solid state drives, or other data storage devices. The data storage device 740 can store program code 740A for person detection, 740B for tracking movement, and/or 740C for generating a report on the tracked movement. Any or all of these program code blocks may be included in a given computing system. The communication subsystem 750 of the computing device 700 may be embodied as any network interface controller or other communication circuit, device, or collection thereof, capable of enabling communications between the computing device 700 and other remote devices over a network. The communication subsystem 750 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 700 may also include one or more peripheral devices 760. The peripheral devices 760 may include any number of additional input/output devices, interface devices, and/or other peripheral devices. For example, in some embodiments, the peripheral devices 760 may include a display, touch screen, graphics circuitry, keyboard, mouse, speaker system, microphone, network interface, and/or other input/output devices, interface devices, and/or peripheral devices.

Of course, the computing device 700 may also include other elements (not shown), as readily contemplated by one of skill in the art, as well as omit certain elements. For example, various other sensors, input devices, and/or output devices can be included in computing device 700, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 700 are readily contemplated by one of ordinary skill in the art given the teachings of the present invention provided herein.

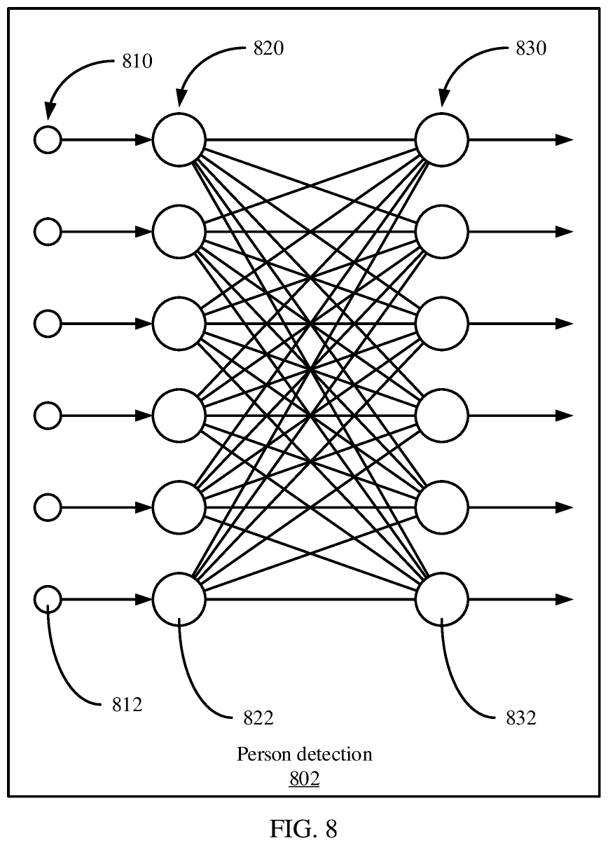

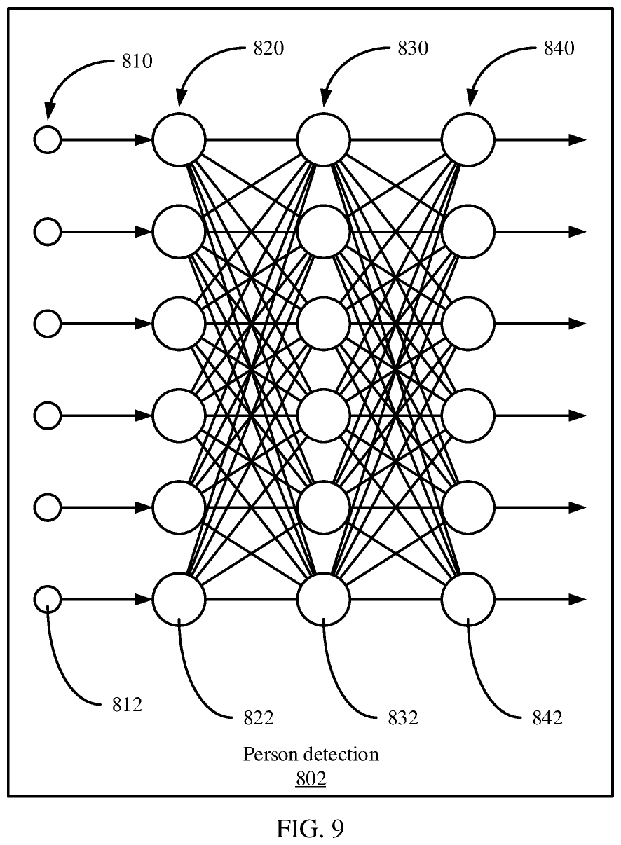

Referring now to FIGS. 8 and 9, exemplary neural network architectures are shown, which may be used to implement parts of the present models, such as the person detection 802. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 820 of source nodes 822, and a single computation layer 830 having one or more computation nodes 832 that also act as output nodes, where there is a single computation node 832 for each possible category into which the input example could be classified. An input layer 820 can have a number of source nodes 822 equal to the number of data values 812 in the input data 810. The data values 812 in the input data 810 can be represented as a column vector. Each computation node 832 in the computation layer 830 generates a linear combination of weighted values from the input data 810 fed into input nodes 820, and applies a non-linear activation function that is differentiable to the sum. The exemplary simple neural network can perform classification on linearly separable examples (e.g., patterns).

A deep neural network, such as a multilayer perceptron, can have an input layer 820 of source nodes 822, one or more computation layer(s) 830 having one or more computation nodes 832, and an output layer 840, where there is a single output node 842 for each possible category into which the input example could be classified. An input layer 820 can have a number of source nodes 822 equal to the number of data values 812 in the input data 810. The computation nodes 832 in the computation layer(s) 830 can also be referred to as hidden layers, because they are between the source nodes 822 and output node(s) 842 and are not directly observed. Each node 832, 842 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 832 in the one or more computation (hidden) layer(s) 830 perform a nonlinear transformation on the input data 812 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method for tracking movement, comprising:
performing person detection in frames from multiple video streams to identify detection images;
combining visual and location information from the detection images to generate scores for pairs of detection images across the multiple video streams and across frames of respective video streams;
generating a pairwise detection graph using the detection images as nodes and the scores as weighted edges; and
changing from a current view of the multiple video streams to a next view of the multiple video streams, responsive to a determination that a score between consecutive frames of the view is below a threshold value and that a score between coincident frames of the current view and the next view is above the threshold value.

2. The method of claim 1, further comprising synchronizing the multiple video streams to identify temporal correspondences between frames of the multiple video streams.

3. The method of claim 1, further comprising extracting the visual information based on a visual similarity between detection images.

4. The method of claim 1, further comprising extracting the location information based on a projection of two-dimensional coordinates into a three-dimensional environment for the detection images and determining a distance between the projected coordinates.

5. The method of claim 1, wherein generating the pairwise detection graph includes determining edges between detection images from different frames of a same video stream and determining edges between detection images from different video streams at corresponding times.

6. The method of claim 1, further comprising performing an action that includes generating a report for a healthcare professional for decision-making related to a patient's treatment, based on tracked movement of the patient.

7. The method of claim 1, wherein the current view includes a detected person within a healthcare facility and wherein the multiple video streams are generated by video cameras within the healthcare facility.

8. The method of claim 1, wherein combining the visual and location information includes adding an output from a visual branch to an output of a location branch.

9. The method of claim 1, wherein changing from the current view to the next view includes changing a display on a user interface device to display the next view on a screen.

10. A system for tracking movement, comprising:
a hardware processor; and
a memory that stores a computer program which, when executed by the hardware processor, causes the hardware processor to:
perform person detection in frames from multiple video streams to identify detection images;
combine visual and location information from the detection images to generate scores for pairs of detection images across the multiple video streams and across frames of respective video streams;
generate a pairwise detection graph using the detection images as nodes and the scores as weighted edges; and
change from a current view of the multiple video streams to a next view of the multiple video streams, responsive to a determination that a score between consecutive frames of the view is below a threshold value and that a score between coincident frames of the current view and the next view is above the threshold value.

11. The system of claim 10, wherein the computer program further causes the hardware processor to synchronize the multiple video streams to identify temporal correspondences between frames of the multiple video streams.

12. The system of claim 10, wherein the computer program further causes the hardware processor to extract the visual information based on a visual similarity between detection images.

13. The system of claim 10, wherein the computer program further causes the hardware processor to extract the location information based on a projection of two-dimensional coordinates into a three-dimensional environment for the detection images and determining a distance between the projected coordinates.

14. The system of claim 10, wherein the computer program further causes the hardware processor to determine edges between detection images from different frames of a same video stream and to determine edges between detection images from different video streams at corresponding times.

15. The system of claim 10, wherein the computer program further causes the hardware processor to perform an action that includes generating a report for a healthcare professional for decision-making related to a patient's treatment, based on tracked movement of the patient.

16. The system of claim 10, wherein the current view includes an individual relates within a healthcare facility and wherein the multiple video streams are generated by video cameras within the healthcare facility.

17. The system of claim 10, wherein the computer program further causes the hardware processor to an output from a visual branch to an output of a location branch to combine the visual and location information.

18. The system of claim 10, wherein changing from the current view to the next view includes changing a display on a user interface device to display the next view on a screen.

19. A method for tracking movement in a healthcare facility, comprising:
performing person detection in frames from multiple video streams in a healthcare facility to identify detection images;
combining visual and location information from the detection images to generate scores for pairs of detection images across the multiple video streams and across frames of respective video streams;
generating a pairwise detection graph using the detection images as nodes and the scores as weighted edges;
tracking movement of a patient based on a selected detection image and a comparison of the scores to a threshold value;
changing from a current view of the multiple video streams on a display device to a next view of the multiple video streams, responsive to a determination that a score between consecutive frames of the view is below a threshold value and that a score between coincident frames of the current view and the next view is above the threshold value; and
generating a report for a healthcare professional for decision-making related to a patient's treatment, based on the tracked movement.

20. The method of claim 19, wherein generating the pairwise detection graph includes determining edges between detection images from different frames of a same video stream and determining edges between detection images from different video streams at corresponding times.
